(19) United States
(12) Patent Application Publication
(10) Pub. No.: US 2002/0187562 A1
(43) Pub. Date: Dec. 12, 2002

(54) Materials for separating and detecting enantiomers of amphetamine or methamphetamine, and their application

(76) Inventors: Ahai-Chang Lua, Hualien City (TW); Tsong-Yung Chou, Hualien City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 09/875,892

(22) Filed: Jun. 8, 2001

(51) Int. Cl. G01N 33/561

(52) U.S. Cl. 436/516

(57) ABSTRACT

The present invention discloses a material for chromatography comprising an antibody capable of differentiating at least an enantiomer of amphetamine or methamphetamine, and a support, wherein the antibody is bonded to the support. The material is capable of being employed to differentiate at least an enantiomer of amphetamine or methamphetamine in a sample. Also disclosed in the present invention are the methods and kits for detecting or identifying at least an enantiomer of amphetamine or methamphetamine in a sample, and methods for separating or purifying at least an enantiomer of amphetamine or methamphetamine from a sample.

### FIELD OF THE INVENTION

The present invention relates to novel materials and methods for separating and detecting at least an enantiomer of amphetamine (AP) or methamphetamine (MA).

### BACKGROUND OF THE INVENTION

Drug abuse cases have been increasing in number and causing a serious social problem in recent years. Illicit drug abuse is not only harmful to individuals' health but also to the security of society. In the field of forensic medicine, therefore, there is an urgent need for developing an easy and reliable method to detect and identify the abused drugs and their metabolites in the human body.

In eastern countries, most drug abuse cases are involved in D-methamphetamine (D-MA, (S)-(+)-N-methyl-1-phenyl-2-aminopropane). The conventional assays to identify drug abuse cases are conducted based on the detection of the presence of MA or AP in urine or blood samples from subjects. However, the disadvantage of the conventional assays is their incapability to differentiate among the enantiomers of AP and/or MA. In view of the fact that the stimulation of L-methamphetamine (L-MA, (R)-(−)-N-methyl-1-phenyl-2-aminopropane) to the central nervous system of humans is much weaker than that of D-MA, L-MA has become a common active ingredient in OTC medicines for alleviating symptoms of common colds, e.g., runny nose. L-MA and L-amphetamine (L-AP) can be found in urine samples from the subjects who have taken certain OTC medicines with regulatory approvals in European countries and the USA. Thus, it is crucial that only the assays capable of differentiating among the enantiomers of AP or MA be employed to identify with accuracy the abuse cases of D-MA. The known easiest way to differentiate among the enantiomers of AP and/or MA is to determine the specific rotation of a sample solution compared to a relevant control. However, this easiest way is valueless in practice since the concentration of metabolites found in the sample from a tested subject is always too low to determine its specific rotation.

In recent years, several approaches have been developed in an attempt to differentiate among the enantiomers of AP and/or MA. To detect the presence of D-MA in those commercial pharmaceuticals on which the packaging has the inclusion of L-MA labeled, the pharmaceuticals were extracted with CH₂Cl₂ and the volatile CH₂Cl₂ was evaporated, followed by the injection of the concentrated extracts into a chiral stationary phase column (HPLC/MS) where N-(3,5-dinitrobenzoyl)phenylglycine is bonded to the substrates packed in the column, as disclosed by Lee et al. (Anal. Chem. 58 (1986), 1349-1352). Lee et al. could detect the presence of D-MA in a pharmaceutical which amount is 100 folds less than the amount of L-MA.

In another approach, MA (or AP) was reacted with isothiocyanate to form derivatives and then the derivatives were separated by a—cyclodextrin stationary phase, as disclosed in Rizzi et al. (Chromatographia. 339 (1994) 131). Lemr et al. (J. Liq. Chrom.&Rel. Technol., 19 (19), 1996, 3173-3191) disclosed the use of a chromatographic column comprising silica gel bonded with -cyclodextrin for separating the enantiomers in the mixture of three compounds of similar structures: ephedrine, MA and selegiline. However, Lemr et al neither suggested nor disclosed the minimum detectable level of the enantiomers of their method. Katagi et al. (J. Chromatogr. B, 676 (1996) 35-43) tried to monitor AP and MA by extracting AP and MA using an ion-exchange resin column and subjecting them to an assay using high performance liquid chromatography (HPLC) apparatus equipped with a thermo-spray mass spectrometry (MS), where the HPLC column was packed with silica gel coated with—cyclodextrin. According to the method of Katagi et al., the minimum detectable concentration was 0.2-0.5 ng/ml in the selected ion monitoring mode and 50.0-100.0 ng/ml in the UV₂₂₀ₙₘ monitoring mode. Although the Katagi et al. method could separate enantiomers of AP and MA, the protocol is complicated and time-consuming. Moreover, in addition to the protonated AP and MA, contaminants would be also obtained after the extraction step. This caused disadvantageous interferenes occurring upon the subsequent HPLC separation step.

Makino et al. (Forensic Science International 78 (1996) 65-70) disclosed the HPLC separation of enantiomers of AP and MA using a column packed with filler material to which a chiral crown ether was bonded. However, the samples had to be concentrated by n-hexane and admixed with 0.05 N HCl before the HPLC separation. In the Hutchaleelaha et al. (J Chromatogr. B 103 (1994) 658) method, AP and MA in serum samples were extracted with ethyl acetate and the enantiomers of AP and MA were reacted with (−)-1-(9-fluorenyl)ethyl chloroformate, prior to the HPLC separation. Using a similar method, Hutchaleelaha et al. (J. Anal. Toxicol. 19 (1995) 139) monitored MA and its metabolites (AP, p-hydroxy-MA and p-hydroxy-AP) in urine samples from the rabbits fed MA.

More complicated approaches have been made in an attempt to detect AP and MA. In the Noggle et al. approaches (J. Forensic Sci. 31 (1986) 732; J. Chromatogr. Sci. 28 (1990) 529; Anal. Chem. 58 (1986) 1643), MA was derivatized with 2,3,4,6-tetra-O-acetyl- -D-glucopyranosylisothiocyanate and the resultant derivatives of the enantiomers of MA was separated using HPLC. AP and MA in urine samples could be extracted by a solid phase extraction and then reacted with N, -(2,4-dinitro-5-fluorophenyl)-L-alaninamide to form diastereomers, which were then analyzed via a reverse-phase chromatograph, as disclosed by Foster et al. (J. Anal. Toxicol. 22 (1998) 265-269). In the Jin et al. approach (Chromatographia 38 (1994) 595), AP and MA were reacted with the asymmetric agent N-trifluoroacetylprolyl chloride to form diastereomers, and the diastereomers were separated via gas chromatography (GC) using a capillary column, the inside of which was coated with 2,6-di-O-pentyl-3-O-propionyl- -cyclodextrin or 2,6-di-O-pentyl-3-O-trifluoroacetyl-β-cyclodextrin. Cody et al. (J. Anal. Toxicol. 17 (1993) 321; J. Anal. Toxicol. 14 (1990) 321; J. Chromatogr. 77 (1992) 580) tried to extract AP and MA from urine samples with chlorobutane and react the extracted AP and MA with S′-(−)-N-trifluoroacetylprolyl chloride to form diastereomers, which were then subjected to a GC-MS analysis. In the Hays et al. approach (J. Chromatogr. 398 (1987) 239), the diastereomers of AP and MA were similarly formed with S′-(−)-N-trifluoroacetylprolyl chloride but then separated via liquid chromatography (LC) with a chiral stationary phase. Nagai et al. (J. Chromatogr. 525 (1990) 203) disclosed the use of two asymmetric stationary phase columns connected serially to separate the derivatives of AP and MA that had been extracted with a benzoyl solvent from mouse urine samples.

Most of the prior techniques as mentioned include protocols where test substances are extracted in a liquid or solid phase and concentrated, followed by the separation using a chiral stationary phase column(s) to which, in most cases, cyclodextrin is bound. Some of the prior techniques include a protocol where test substances (AP or MA) are reacted with a chiral reagent to form diastereomers, and the diastereomers are then separated using an achiral stationary phase column(s). In the latter case, the employed chiral reagent must be present in a highly pure form. Otherwise, undesired diastereomers would form. This makes it more difficult to differentiate SR ′ and RS′ diastereomers in a chromatographic analysis using an achiral stationary phase column(s), since both kinds of diastereomers have the same profiles in such a chromatographic analysis. Given the foregoing, it is very difficult to quantify D-MA in a trace amount if it is present in a legal, commercial medicament containing L-MA. Also, the derivation reactions that have to be conducted before analyses not only cause an increase of expenditure of reagents and time, but also a reduction of sensitivity of detection.

All the methods used for the detection of AP or MA mentioned in the literature (e.g., J. Forensic Science International, 96 (1998) 61-70) are to pretreat the urine samples with extraction (liquid or solid phase) or chemical derivation prior to the analysis analyzed via chromatography. Nevertheless, with the adverse factors of the existing methods of detecting AP or MA taken into consideration, i.e., time-consuming, greater expense, necessary intervention by technicians and reduced sensitivity, directly analyzing samples from drug abuse suspects for D-MA and D-AP with results accurate enough to use as evidence in ascertaining whether illegal drugs were abused is desired.

Accordingly, there is a need for a method that can rapidly detect or separate at least an enantiomer of AP or MA with ease and accuracy.

### SUMMARY OF THE INVENTION

An object of the present invention is to provide a material for chromatography capable of being employed to differentiate at least an enantiomer of AP or MA. The material comprises an antibody capable of differentiating at least an enantiomer of AP or MA and a support where the antibody is bonded to the support.

Another object of the present invention is to provide a process for preparing a material for chromatography capable of differentiating at least an enantiomer of AP or MA. The process comprises allowing an antibody capable of differentiating at least an enantiomer of AP or MA to bond to a support.

Another object of the present invention is to provide a column for chromatography capable of being employed to differentiate at least an enantiomer of AP or MA. The column comprises, as at least part of the packing material, a material comprising an antibody capable of differentiating at least an enantiomer of AP or MA and a support where the antibody is bonded to the support.

Another object of the present invention is to provide a method of separating at least an enantiomer of AP or MA from a sample, which comprises subjecting the sample to chromatography using a material capable of differentiating at least an enantiomer of AP or MA as at least part of the packing material and separating the differentiated enantiomer of AP or MA. The material capable of differentiating at least an enantiomer of AP or MA comprises an antibody capable of differentiating at least an enantiomer of AP or MA and a support where the antibody is bonded to the support.

Another object of the present invention is to provide a method of purifying at least an enantiomer of AP or MA from a sample, which comprises subjecting the sample to chromatography using a material capable of differentiating at least an enantiomer of AP or MA as at least part of the packing material and purifying the differentiated enantiomer of AP or MA. The material capable of differentiating at least an enantiomer of AP or MA comprises an antibody capable of differentiating at least an enantiomer of AP or MA and a support where the antibody is bonded to the support.

Another object of the present invention is to provide a method of identifying at least an enantiomer of AP or MA in a sample, which comprises subjecting the sample to chromatography using a material capable of differentiating at least an enantiomer of AP or MA as at least part of the packing material, and identifying the differentiated enantiomer of AP or MA. The material capable of differentiating at least an enantiomer of AP or MA comprises an antibody capable of differentiating at least an enantiomer of AP or MA and a support where the antibody is bonded to the support.

Another object of the present invention is to provide a method of detecting at least an enantiomer of AP or MA in a sample, which comprises subjecting the sample to chromatography using a material capable of differentiating at least an enantiomer of AP or MA as at least part of the packing material, and determining the presence or absence of the enantiomer of AP or MA. The material capable of differentiating at least an enantiomer of AP or MA comprises an antibody capable of differentiating at least an enantiomer of AP or MA and a support where the antibody is bonded to the support.

Another object of the present invention is to provide a kit for detecting or identifying at least an enantiomer of AP or MA in a sample, which comprises a means containing a material capable of differentiating at least an enantiomer of AP or MA, wherein the means is provided for a thin layer chromatograph (TLC). The material capable of differentiating at least an enantiomer of AP or MA comprises an antibody capable of differentiating at least an enantiomer of AP or MA and a support where the antibody is bonded to the support.

### BRIEF DESCRIPTION OF THE FIGURES

FIG. 1 is a schematic diagram of an overall system configuration of liquid chromatography according to an embodiment of the present invention.

FIG. 2 is a schematic diagram of an overall system configuration of high-performance liquid chromatography according to an embodiment of the present invention.

FIG. 3 is a chromatograph resulting from analyses of samples using the liquid chromatography according to an embodiment of the present invention.

FIG. 4 is a chromatograph resulting from analyses for the enantiomers of AP using high-performance liquid chromatography according to an embodiment of the present invention.

FIG. 5 is a chromatograph resulting from analyses for the enantiomers of MA using high-performance liquid chromatography according to an embodiment of the present invention.

FIG. 6 is a chromatograph resulting from analyses of urine samples using the high-performance liquid chromatography according to the embodiment of the present invention.

FIG. 7 is a chromatograph resulting from the extraction and separation of the enantiomers of racemic MA using the high-performance liquid chromatography according to the embodiment of the present invention.

### DETAILED DESCRIPTION OF THE PREFERRED EMBODIMENTS

The term “sample” means a sample that comprises or is supposed to comprise any enantiomer(s) of AP or MA. Preferably, the term “sample” refers to a sample to be detected or identified for the presence of at least an enantiomer of AP or MA, or a sample containing a mixture of more than one enantiomer of AP or MA to be separated or purified for at least an enantiomer of AP or MA. More preferably, the term “sample” refers to a biological sample including serum, urine or the like.

The present invention provides, as at least part of the packing material in a column employed for chromatography, a material comprising an antibody capable of differentiating at least an enantiomer of AP or MA that is bonded to a support. The support that can be employed in the present invention may be solid or colloidal. For example, a colloidal support may be a polysaccharide or a silica gel. The column filled partly or completely with the material according to the present invention, which constitutes a part of the present invention, may be employed for liquid chromatography (LC) or high-performance liquid chromatography (HPLC). Column chromatography inclusive of LC and HPLC is one of the most widely used techniques in analytical work. The general principles, protocols, equipment and precautions to prepare and run columns for dealing with the various types of chromatographic separations are well known in the art.

In an embodiment of the present invention, a urine sample in an appropriate amount may be directly loaded onto the column according to the present invention. Every enantiomer of AP and/or MA that is present in the sample can be bound to the material according to the present invention. The mobile phase employed to elute the bound enantiomer(s) is a buffer system. As the affinity of the employed antibody varies depending upon the particular type of enantiomers of AP and MA, the elution buffer (mobile phase) can override the bound enantiomer(s) and gradually elute them from the column. Different enantiomers can be removed from the column by changing the pH, ion strength or polarity of the mobile phase. The effluent from the column may be collected in a series of test tubes, most commonly using an automatic fraction collector. This apparatus collects a preset volume or number of drops of the effluent into a test tube before moving the next tube into position. Each fraction is then analyzed for the presence of the enantiomer(s) of interest. The content of each fraction can be monitored continuously by subjecting the effluent to a quantitative assay, such as a mass detector.

In a preferred aspect of the present invention, the antibody employed may be specific to D-MA, preferably a monoclonal antibody specific to D-MA. The antibody may be bonded to silica gel that is employed as at least part of the packing material in a column for a HPLC. Where a urine sample in an appropriate amount is loaded onto the HPLC column, any of the four enantiomers D-MA, L-MA, D-AP and L-AP, if present in the sample, can be bound to the material. As the affinity of the employed antibody varies depending upon the particular type of enantiomers of AP and MA, the bound enantiomer(s) can be gradually eluted from the column by changing the pH, ion strength or polarity of the mobile phase. Therefore, the column of the present invention may be employed to differentiate or further extract at least an enantiomer of AP or MA. The column of the present invention is especially useful in assaying a urine sample from a suspect to identify whether a case of illicit drug (D-MA or D-AP) abuse exists.

The present invention also relates to a method differentiating at least an enantiomer of AP or MA from others. Different enantiomers can be differentiated using the method according to the present invention where a proper antibody is chosen and employed. In addition to the detection or identification for drug abuse cases, the present invention is also useful in purifying a medical-grade enantiomer of AP or MA where the amount of the employed antibody adequately increases. For example, a preparativeHPLC column filled with the material to which an antibody specific to at least an enantiomer of AP or MA is bonded can be prepared according to the present invention.

The present invention provides a material capable of being employed to differentiate at least an enantiomer of AP or MA. The material comprises an antibody capable of differentiating at least an enantiomer of AP or MA and a support. Preferably, the support employed in the present invention is a solid support. More preferably, the solid support may be a polysaccharide, SiO₂, a glass bead, Al₂O₃ or an activated carbon. In embodiments of the present invention, the solid support is a polysaccharide or SiO₂.

The material according to the present invention capable of being employed to differentiate at least an enantiomer of AP or MA is suited to separate, purify, identify or detect at least an enantiomer of AP or MA where the enantiomer is present in a sample.

The present invention also provides a process of preparing a material capable of being employed to differentiate at least an enantiomer of AP or MA. The process comprises allowing an antibody capable of differentiating at least an enantiomer of AP or MA to be bonded to a support. Preferably, the support employed in the present invention is a solid support. More preferably, the solid support may be a polysaccharide, SiO₂, a glass bead, Al₂O₃ or an activated carbon. In the embodiments of the present invention, the solid support is a polysaccharide or SiO₂. An appropriate antibody can be bonded to a solid support, such as a polysaccharide, silica gel or a fine glass bead, using a coupling agent (such as a silane derivative) or a bifunctional compound (such as glutaraldehyde). The utilization of a coupling agent or a bifunctional compound to allow an antibody to be bonded to a support is well known by persons of ordinary skill in the art.

The present invention also provides a column for chromatography capable of being employed to differentiate at least an enantiomer of AP or MA. The column comprises, as at least part of the packing material, a material that comprises an antibody capable of differentiating at least an enantiomer of AP or MA, and a support. Preferably, the material of the present invention comprises a solid support to which an antibody is bonded. More preferably, the solid support may be a polysaccharide, SiO₂, a glass bead, Al₂O₃ or an activated carbon. In the preferred aspects of the present invention, the solid support may be a polysaccharide or SiO₂. In more preferred aspects of the present invention, the solid support to which an appropriate antibody is bonded may be packed manually in a glass column or packed in a steel column with a high-pressure filling means. The column according to the present invention may comprise the material of the present invention as the packing material or part of the packing material.

The proportion of the material of the present invention to the packing material depends upon the scale at which the column chromatograph is intended to run. For example, where the chromatograph is run at a preparative scale, the capacity of the packing material should be such adequate that a workable quantity of a specific enantiomer can be obtained in the chromatograph. Where the chromatograph is run at an analytical scale (e.g., analysis of a sample), the capacity of the packing material is required to detect the presence of a specific enantiomer in the sample in an amount as small as possible. Persons skilled in the art would readily understand and adjust the proportion of the material of the present invention to the packing material depending upon their goals to run a column chromatograph.

The present invention also provides a method of separating at least an enantiomer of AP or MA from a sample, which comprises subjecting the sample to a chromatograph using a material capable of differentiating the enantiomer of AP or MA as at least part of the packing material, and separating the differentiated enantiomer of AP or MA. Preferably, the chromatography is HPLC, LC or TLC. In an embodiment of the present invention, the method can be employed to separate a specific enantiomer of AP or MA from a sample containing a mixture of enantiomers of AP and MA.

The present invention also provides a method of purifying at least an enantiomer of AP or MA from a sample, which comprises subjecting the sample to a chromatograph using a material capable of differentiating the enantiomer of AP or MA as at least part of the packing material, and purifying the differentiated enantiomer of AP or MA. Preferably, the chromatograph is a HPLC, an LC or a TLC. In an embodiment of the present invention, the method can be employed to purify a specific enantiomer of AP or MA from a sample containing a mixture of enantiomers of AP and MA.

The present invention also provides a method of identifying at least an enantiomer of AP or MA in a sample, which comprises subjecting the sample to a chromatograph using a material capable of differentiating the enantiomer of AP or MA as at least part of the packing material, and identifying the differentiated enantiomer of AP or MA. Preferably, the chromatography is HPLC, LC or TLC. In an embodiment of the present invention, the method can be employed to identify a specific enantiomer of AP or MA in a urine sample containing the enantiomer.

The present invention also provides a method of detecting at least an enantiomer of AP or MA in a sample, which comprises subjecting the sample to chromatography using a material capable of differentiating the enantiomer of AP or MA as at least part of the packing material, and determining the presence or absence of the enantiomer of AP or MA. Preferably, the chromatography is HPLC, LC or TLC. In a preferred aspect of the present invention, the method may be employed to detect at least a specific enantiomer of AP or MA in a urine sample containing the enantiomer(s).

It is also contemplated in the present invention that a kit containing the material of the present invention is useful in detecting or identifying at least an enantiomer of AP or MA in a sample. Preferably, the material capable of differentiating at least an enantiomer of AP or MA as employed in the kit is applied to a means provided for thin layer chromatography (TLC).

Where it is desired to simultaneously detect the presence of more than one enantiomer of AP and MA (e.g., D-AP and D-MA) in a sample, the material of the present invention may contain only one type of antibody. As will be demonstrated in the following examples, the material containing one type of antibody may be sufficient where it is employed to differentiate multiple enantiomers of both AP and MA. Alternatively, the material of the present invention may contain more than one type of antibody (e.g., two types of antibodies). In the alternative case, the specificity and affinity of the employed antibodies differ with respect to each other. The material containing more than one type of antibody, a column filled with the material, a method using the material and a kit containing the material are very useful in determining whether a drug abuse case exists, where more than one illicit drug or the metabolites thereof may be detected in a (blood or urine) sample from the suspect. Therefore, the material containing more than one type of antibody, the columns filled with the material, the methods using the material and the kits constitute parts of the present invention.

All of the literature and publications as recited in the context of the present disclosure are incorporated herein by reference.

Other features and advantages of the present invention will be apparent from the following description of the preferred embodiments and from the claims.

### EXAMPLES

The following examples illustrate various aspects of the present invention but do not limit the claims in any manner whatsoever.

### Example 1

Preparation and Utilization of Polysaccharides Bonded with Antibodies

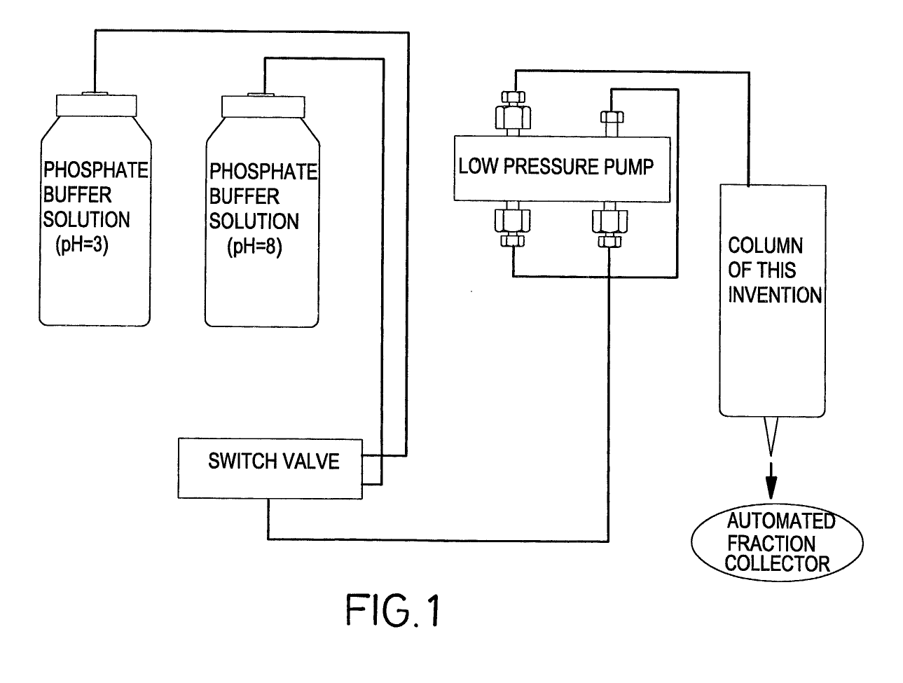

Polysaccharides were activated with CNBr, and then reacted with the antibodies specific to D-MA in an ice bath for 24 hours. After filtration, the colloidal material obtained was washed with phosphate buffered saline. The resultant polysaccharides bonded with the antibodies (Material A) were packed manually in a glass column (internal radius: 1.0 cm, length: 15.0 cm) for an LC. The LC was equipped and conducted as shown in FIG. 1.

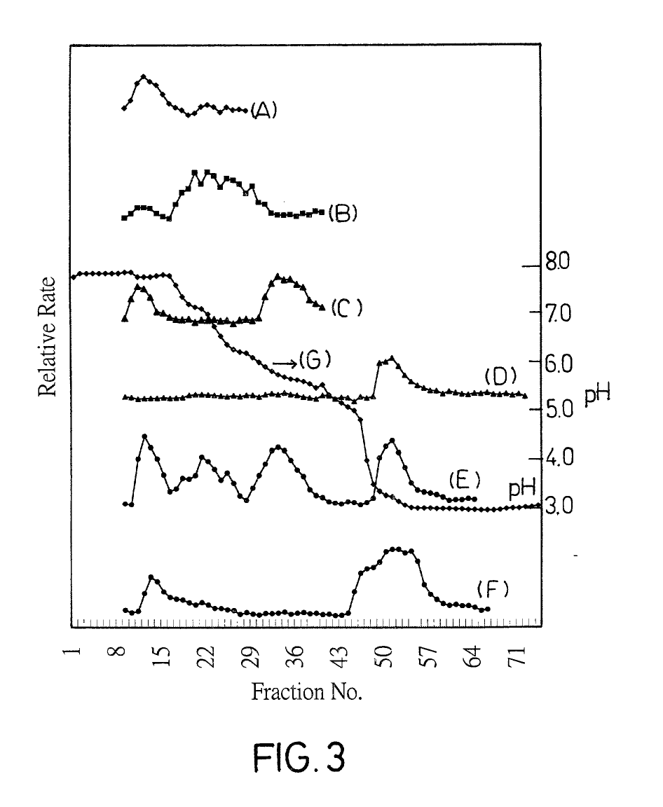

The capability of the LC column in the differentiation of enantiomers of AP and MA was analyzed by directly loading various samples onto the column. The mobile phase was 100.0 mM phosphate buffered saline. The fractions were collected by an automatic collector where the volume of effluent in each fraction 0.50 mL. The fractions were analyzed using an enzyme immunoassay (EIA). The results obtained are depicted in FIG. 3. In FIG. 3, the curve labeled (A) represents a sample containing L-AP, the curve labeled (B) represents a sample containing D-AP, the curve labeled (C) represents a sample containing L-MA, the curve labeled (D) represents a sample containing D-MA, the curve labeled (E) represents a sample containing L-AP, D-AP, L-MA and D-MA, the curve labeled (F) represents a urine sample containing D-MA and the curve labeled (G) shows progressive changes of the pH with the fraction number.

As shown in FIG. 3, the employed material comprising an antibody specific to D-MA was capable of differentiating the enantiomers of both AP and MA. According to FIG. 3, the urine sample was determined with ease to contain D-MA according the LC of the present invention as compared with the profiles of the individual enantiomers of AP and MA

### Example 2

Preparation and Utilization of Silica Gel Bonded with Antibodies

2.1 Modification of the Surface of Silica Gel with a Coupling Agent

In toluene, approximately 5.0 grams of silica gel were suspended and then 5.0 ml of γ-aminopropyltrimethoxysilane were added. The reaction was conducted at 110° C. for 16 hours. After filtration, the gel was washed subsequently with toluene, methanol and ether, and dried under a vacuum condition.

2.2. Bonding of Antibodies to the Modified Silica Gel

In this example the modified silica gel was further treated in two separate reactions to allow two types of antibodies with different specificities to bond to the surface of the silica gel.

Approximately 4.0 grams of the silica gel modified with the coupling agent were suspended in 10.0 ml of phosphate buffered saline, pH 7.0. The suspension was poured into phosphate buffered saline with 5% glutardialdehyde, pH 7.0, and the reaction was allowed to continue for 4 hours. After filtration, the silica gel was washed several times with phosphate buffered saline (pH 7.0).

The resultant silica gel was added to 10.0 ml of phosphate buffered saline (pH 7.0) supplemented with an antibody in the concentration of 1.0 mg/ml that was specific to either D-AP or D-MA. Each reaction was allowed to continue in an ice bath for 16 hours. After filtration, the silica gel with the antibody was washed several times with phosphate buffered saline (pH 7.0).

The resultant solids were added to phosphate buffered saline (pH 7.0) with 5% glycine and then placed in an ice bath for 16 hours. After filtration and then washing several times with phosphate buffered saline (pH 7.0), two kinds of the silica gel bonded with different antibodies were obtained, i.e., the silica gel bonded with the antibody specific to D-AP (Material B) and the silica gel bonded with the antibody specific to D-MA (Material C).

2.3. Preparation and Application of HPLC Columns for Enantiomer Separation

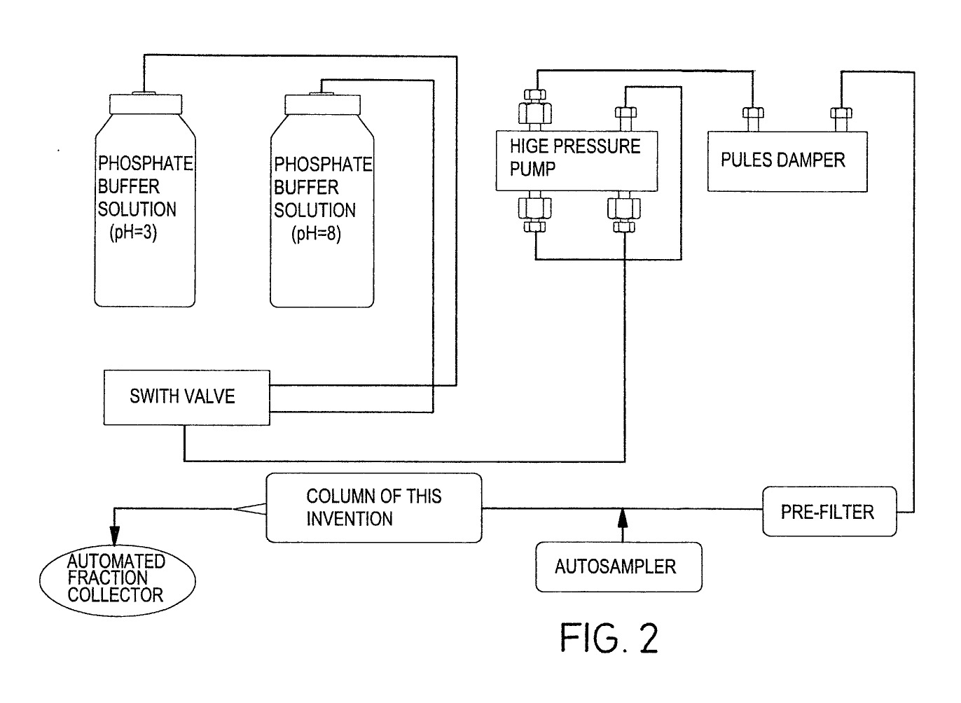

Each silica gel bonded with the antibody obtained in Example 2.2 was admixed with other proper materials to form slurries. The slurries were packed in a steel column (internal radius: 4.6 mm, length: 25.0 cm) using a high-pressure filling means to prepare a HPLC column. The HPLC was equipped and conducted as illustrated in FIG. 2.

2.4 Results

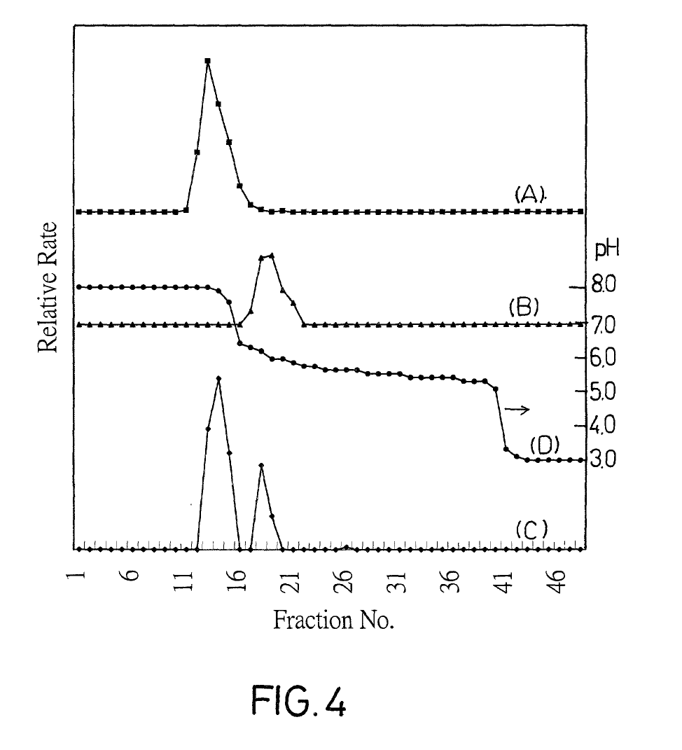

The capability of the HPLC using the steel column containing Material B in separating the enantiomers of AP was analyzed by directly loading various samples onto the column. The mobile phase was 50.0 mM phosphate buffered saline and the flow rate was 0.4 ml/minute. The fractions were collected by an automatic collector where the effluent was collected into each test tube at an interval of 1.5 minutes. The fractions were analyzed using an EIA. The results obtained are depicted in FIG. 4. In FIG. 4, the curve labeled (A) represents a sample containing L-AP, the curve labeled (B) represents a sample containing D-AP, the curve labeled (C) represents a sample containing L-AP and D-AP, and the curve labeled (D) shows progressive changes of the pH with the fraction number. As shown in FIG. 4, the curve labeled (C) clearly indicated that the column containing Material B can separate L-AP and D-AP completely.

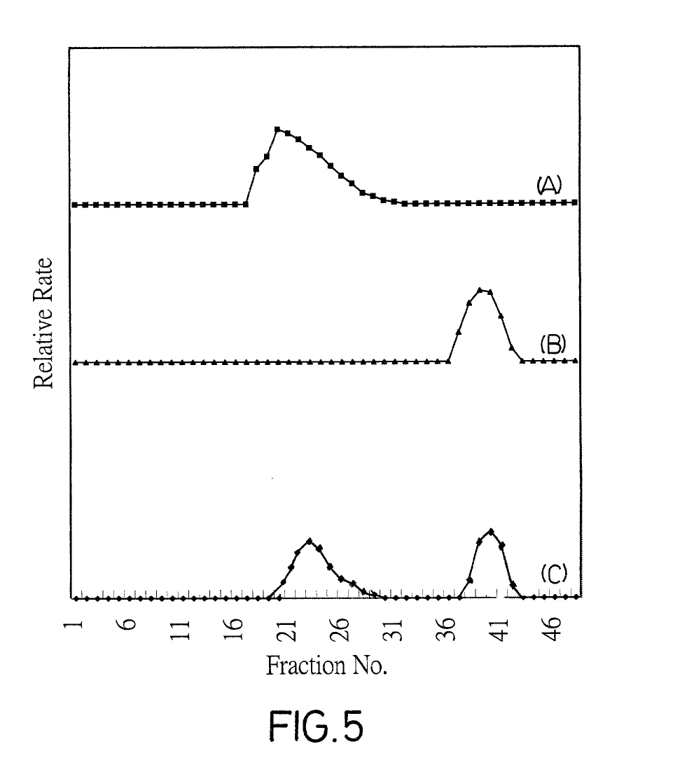

The capability of the HPLC using the steel column containing Material C in separating the enantiomers of MA was analyzed as described above for the HPLC using Material B. The results obtained are depicted in FIG. 5. In FIG. 5, the curve labeled (A) represents a sample containing L-MA, the curve labeled (B) represents a sample containing D-MA, and the curve labeled (C) represents a sample containing L-MA and D-MA. As shown in FIG. 5, the curve labeled (C) clearly indicated that the column containing Material B can separate L-MA and D-MA completely.

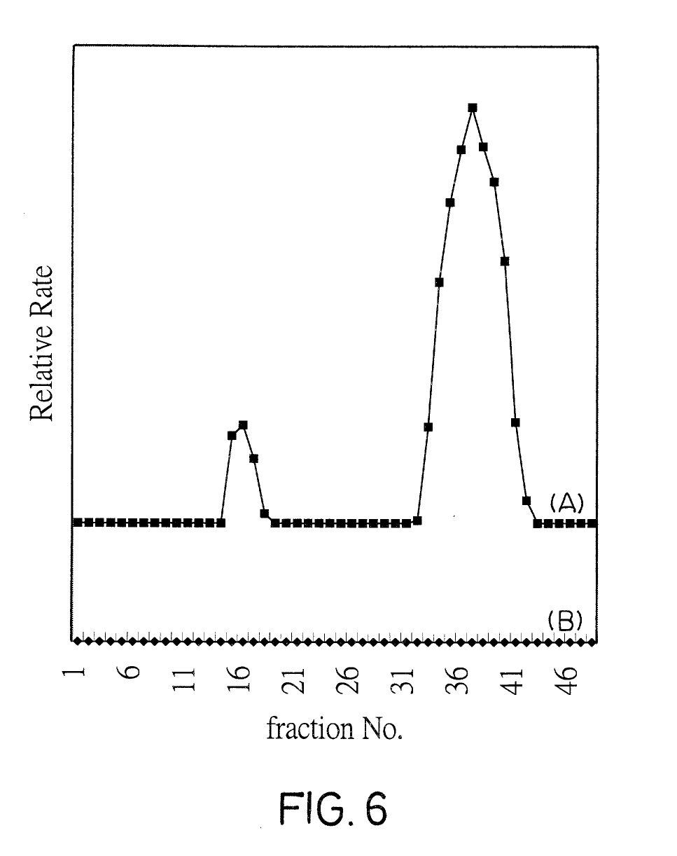

A urine sample was subjected to the HPLC analysis using the steel column containing Material C as described above. The results obtained are depicted in FIG. 6. In FIG. 6, the curve labeled (A) represents a urine sample with D-MA, and the curve labeled (B) represents a urine sample without D-MA. As compared with the curves depicted in FIG. 5, the curve labeled (A) in FIG. 6 clearly indicated that the urine sample analyzed contains D-MA.

### Example 3

Preparative Separation of Enantiomers of MA

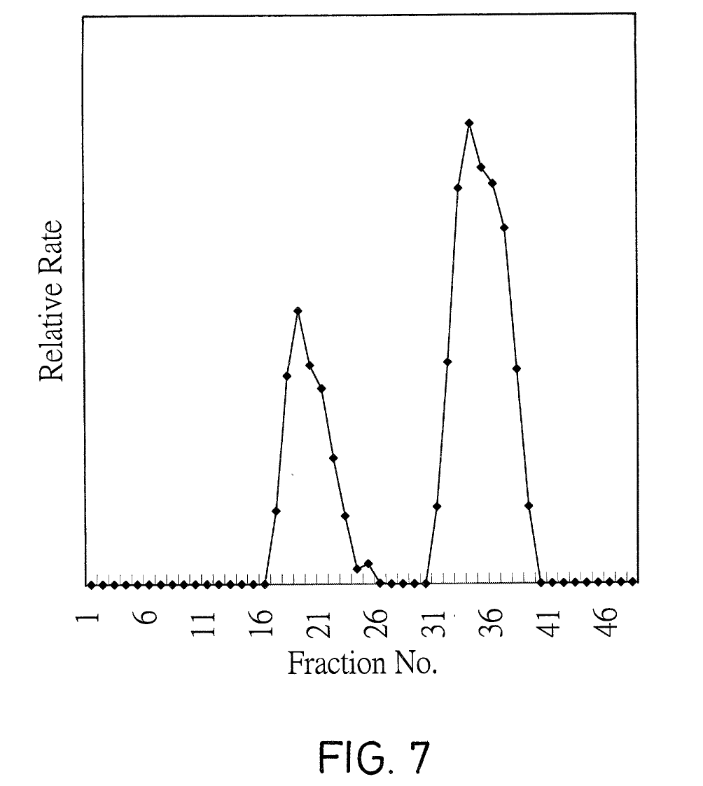

A preparative separation of the enantiomers of MA in a sample was conducted by the HPLC using the steel column containing Material C as described in Example 2. In the sample, the concentration of D-MA and L-MA was adjusted to 1.0 ng/ml. To prepare the sample, the two enantiomers were dissolved in phosphate buffered saline with a constant pH of 8.0. 100.0 microliters of the sample were injected onto the column every 5 minutes for twenty times. Upon elution, the mobile phase was 50.0 mM phosphate buffered saline and the flow rate was 0.4 ml/minute. After the injection, the pH of the elution buffer was changed gradually according to the curve (D) as depicted in FIG. 4. The fractions were collected by an automatic collector where the effluent was collected in each test tube at an interval of 1.5 minutes. The fractions were analyzed using an EIA. The results of the separation are depicted in FIG. 7.

As shown in FIG. 7, D-MA and L-MA in trace amounts in a sample could be extracted and completely separated in view of the two independent peaks from the analyses of fractions.

Various modifications and variations of the present invention will be apparent to those persons skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

We claim

1. A material for chromatography comprising
an antibody capable of differentiating at least an enantiomer of amphetamine or methamphetamine and a support, which is capable of being employed to differentiate at least an enantiomer of amphetamine or methamphetamine in a sample,
wherein the antibody is bonded to the support.

2. A material according to claim 1, wherein the support is a solid support.

3. A material according to claim 2, wherein the solid support is selected from the group consisting of a polysaccharide, SiO₂, a glass bead, Al₂O₃ and active carbon.

4. A material according to claim 3 for use in the separation of at least an enantiomer of amphetamine or methamphetamine from the sample.

5. A material according to claim 3 for use in the purification of at least an enantiomer of amphetamine or methamphetamine from the sample.

6. A material according to claim 3 for use in the identification of at least an enantiomer of amphetamine or methamphetamine in the sample.

7. A material according to claim 3 for use in the detection of the presence or absence of at least an enantiomer of amphetamine or methamphetamine in the sample.

8. A method of detecting and differentiating at least an enantiomer of amphetamine or methamphetamine in a sample, which comprises
subjecting the sample to a chromatograph using a material capable of differentiating at least an enantiomer of amphetamine or methamphetamine as at least part of the packing material, and determining the presence of the enantiomer(s) of amphetamine or methamphetamine, wherein the material capable of differentiating at least an enantiomer of amphetamine or methamphetamine comprises an antibody capable of differentiating at least an enantiomer of amphetamine or methamphetamine and a support where the antibody is bonded to the support.

9. A method according to claim 8 for use in the separation of at least an enantiomer of amphetamine or methamphetamine from the sample.

10. A method according to claim 8 for use in the purification of at least an enantiomer of amphetamine or methamphetamine from the sample.

11. A method according to claim 8 for use in the identification of at least an enantiomer of amphetamine or methamphetamine in the sample.

12. A method according to claim 8 for use in the detection of the presence or absence of at least an enantiomer of amphetamine or methamphetamine in the sample.

13. A method according to claim 8 wherein the sample is a biological sample.

14. A method according to claim 13 wherein the biological sample is a urine sample from a subject.

15. A method according to claim 14 wherein the sample is directly subjected to the chromatography.

16. A kit for detecting or identifying at least an enantiomer of amphetamine or methamphetamine in a sample, which comprises a means containing a material capable of differentiating at least an enantiomer of amphetamine or methamphetamine, wherein
the means is provided for a thin layer chromatography and
the material capable of differentiating at least an enantiomer of amphetamine or methamphetamine comprises an antibody capable of differentiating at least an enantiomer of amphetamine or methamphetamine and a support where the antibody is bonded to the support.

17. A kit according to claim 16 wherein the biological sample is a urine sample from a subject.
